(19) United States
(12) Patent Application Publication
(10) Pub. No.: US 2022/0401088 A1
(43) Pub. Date: Dec. 22, 2022

(54) METHOD FOR CONTROLLING AN ARTICULATING INSTRUMENT

(71) Applicant: Titan Medical Inc., Toronto (CA)

(72) Inventors: Jesse Thomas Dean, Raleigh, NC (US); Perry A. Genova, Chapel Hill, NC (US)

(21) Appl. No.: 17/807,661

(22) Filed: Jun. 17, 2022

Related U.S. Application Data
(60) Provisional application No. 63/212,716, filed on Jun. 20, 2021.

(51) Int. Cl. A61B 17/00; A61B 34/37

(52) U.S. Cl. CPC A61B 17/00234; A61B 34/37; A61B 2034/302

(57) ABSTRACT

A method for controlling an articulating surgical instrument is disclosed. The instrument includes a manipulator and a positioner actuable to position a distal segment within an instrument workspace. The manipulator is attached to the distal segment and includes a distal end configured for mounting an operational tool for performing an operation within the instrument workspace, the manipulator being actuable to manipulate the distal end of the manipulator. The method involves receiving input including position input signals representing a position within an input workspace and orientation input signals representing an orientation within the input workspace and causing generating position control signals for actuating the positioner to move the distal segment within the instrument workspace to a physical position represented by the position input signal and generating manipulation control signals based on the orientation input signals for actuating the manipulator to orient the distal end within the instrument workspace.

### TECHNICAL FIELD

This disclosure relates generally to a surgical instrument apparatus for performing a surgical procedure within a body cavity of a patient.

### DESCRIPTION OF RELATED ART

Robotic surgery systems commonly employ one or more articulating instruments to perform surgical functions within a surgical site in a body cavity of a patient. The articulating instruments may be controlled by a processor circuit that receives inputs from an input device having some means of sensing movements of a surgeon's hands. For example, the input device may include a pair of hand controllers that are grasped in the surgeon's hand and moved to cause corresponding movement of the articulating instruments. It is generally desirable that the movement of the articulating instruments closely mimic the surgeon's hand movements so that performing operations in the surgical site is intuitive.

In commonly owned PCT patent publication WO2016176755A1 filed on Dec. 15, 2015, a method for controlling a dexterous tool in a robotic control system is disclosed. An input device having a handle capable of translational and rotational movement is used to control a tool positioning device of the dexterous tool to position and orient an end effector in response to the position and orientation of the handle. In this control method the location of the end effector is thus determined by the position of the handle if the input device. A processor circuit performs inverse kinematic transformations on the position of the end effector to generate actuation signals for the tool positioner to cause the end effector to move to a physical position corresponding to the position and orientation of the handle. The control of the tool positioning device is intuitive in the sense that the position and orientation of the end effector is substantially similar to the position and orientation of the surgeon's hands. However, by taking the end effector as the point of focus for controlling the tool positioner, the location and position of other portions of the tool positioner are not controlled by the surgeon, but rather calculated by the processor circuit to position the end effector. There remains a need for a mode of controlling a tool positioner that provides the surgeon with control over other portions of the tool positioner in addition to controlling the position of the end effector.

### SUMMARY

In accordance with one disclosed aspect there is provided a method for controlling an articulating surgical instrument. The instrument includes a manipulator and a positioner, the positioner being actuable to position a distal segment of the positioner within an instrument workspace. The manipulator is attached to the distal segment of the positioner and includes a distal end configured for mounting an operational tool for performing an operation within the instrument workspace, the manipulator being actuable to manipulate the distal end of the manipulator within the instrument workspace. The method involves receiving input signals at a processor circuit, the input signals including position input signals representing a position within an input workspace, and orientation input signals representing an orientation within the input workspace. The method also involves causing the processor circuit to generate position control signals for actuating the positioner to move the distal segment within the instrument workspace to a physical position represented by the position input signals. The method further involves causing the processor circuit to generate manipulation control signals based on the orientation input signals for actuating the manipulator to orient the distal end within the instrument workspace.

Receiving input signals may involve receiving input signals from an autonomous controller processor circuit operably configured to autonomously generate the position input signals and the orientation input signals.

Receiving input signals may involve generating the input signals in response to movements of an operator's hand.

Generating the input signals in response to movements of an operator's hand may involve at least one of receiving movement signals from a sensor disposed to monitor free movements of an operator's hand within an input region, receiving movement signals from a movement sensor grasped or attached to the operator's hand, the movement sensor being responsive to free movements of the operator's hand, or receiving movement signals from a virtual reality headset worn by the operator.

The method may involve causing the processor circuit to process the movement signals to generate the position input signals and orientation input signals.

Causing the processor circuit to process the movement signals may involve filtering the free movements of the operator's hand to extract movements of the operator's digits with respect to one of the palm of the operator's hand or the wrist of the operator's hand.

Generating the input signals may involve generating the position input signals in response to translations of the operator's hand in three translational degrees of freedom within the input workspace, and generating the orientation input signals in response to rotations of the operator's hand in at least two rotational degrees of freedom within the input workspace.

Generating the position input signals may involve kinematically transmitting translational movements of the operator's hand in three translational degrees of freedom via a first kinematic structure to a plurality of encoders operable to produce translational movement signals, and processing the translational movement signals to generate the position input signals.

Generating the orientation input signals may involve kinematically transmitting orientation movements of the operator's hand in the at least two rotational degrees of freedom via a second kinematic structure to a plurality of encoders operable to produce orientation signals, and processing the orientation signals to generate the orientation input signals.

The positioner may include a first plurality of segments extending between a bulkhead segment and the distal segment, the first plurality of segments being selectively actuable by transmitting actuation forces via a first plurality of control wires extending through the first plurality of segments, and actuating the positioner may involve generating the position control signals to selectively actuate the first plurality of control wires to cause respective movements of the first plurality of segments to position the distal segment at the physical position.

The manipulator may include a second plurality of segments extending between the distal segment of the positioner and the distal end of the manipulator, the second plurality of segments being moveable in response to transmitting actuation forces delivered via a second plurality of control wires extending through the first plurality of segments and through the second plurality of segments, and actuating the manipulator may involve generating the manipulator control signals to selectively actuate the second plurality of control wires to cause respective movements of the second plurality of segments to orient the distal end within the instrument workspace.

The first plurality of segments may include a plurality of adjacently stacked vertebra extending between the bulkhead segment and the distal segment and the control wires may be coupled to the distal segment and selectively actuating the first plurality of control wires may involve actuating the first plurality of control wires to cause the distal segment to move to position the distal segment at the physical position, and each vertebra may be coupled to move in at least one of a pitch axis and a yaw axis with respect to adjacent vertebra and the actuation forces delivered via the first plurality of control wires may cause the respective vertebra to be angled with respect to each other to bend the instrument in a continuous arc.

The first plurality of segments may include a plurality of elongate segments coupled together by respective joints.

The positioner may be coupled to a rigid shaft and wherein the position input signals may include an insertion depth position within the input workspace, and actuating the positioner to move the distal segment within the instrument workspace may involve causing the rigid shaft to be advanced or retracted in response to the insertion depth position.

The rigid shaft may be received in a bore of an insertion tube and the instrument workspace may extend outwardly from an end of the insertion tube.

Causing the rigid shaft to be retracted may involve causing the rigid shaft to be retracted such that the distal segment of the positioner is disposed proximate the end of the insertion tube and the manipulator remains extending outwardly from the end of the insertion tube and remains capable of movement with respect to the distal segment.

The method may involve generating a positioner operational envelope defining boundaries to movement of the distal segment of the positioner within a portion of the instrument workspace, and generating an alert signal when the position input signals represent a position in the instrument workspace that lie on or outside the positioner operational envelope.

Receiving input signals may involve causing an input device to generate the input signals in response to movements of an operator's hand and may further involve delivering haptic feedback via the input device to the operator's hand in response to the alert signal.

The method of #generating the positioner operational envelope may involve generating the positioner operational envelope in the instrument workspace and mapping the positioner operational envelope from the instrument workspace to the input workspace and generating the alert signal may involve generating the alert signal when the position input signals represent a position in the input workspace that lies on or is outside the positioner operational envelope mapped to the input workspace.

The processor circuit may be operably configured to generate display signals for displaying a view of the instrument workspace and generating the display signals may involve generating display signals including an overlay image corresponding to the positioner operational envelope.

The positioner operational envelope may involve an insertion/retraction region that represents a region of the instrument workspace within which the positioner should be constrained in a straightened condition for insertion or retraction of the instrument into or out of the instrument workspace.

The positioner operational envelope may further involve a free movement region that represents a region of the instrument workspace within which the positioner is able to move after the positioner is disposed outside of the insertion/retraction region of the positioner operational envelope.

The articulating instrument may include one of a set of articulating instruments of differing instrument types used in a surgical procedure and the positioner operational envelope may include at least one of one of a pre-defined positioner operational envelope for all differing instrument types, a pre-defined positioner operational envelope selected based on the instrument type, a positioner operational envelope based at least in part on surgical data associated with a target operational site, a positioner operational envelope based at least in part on surgical data provided by a scan of the target operational site, or a positioner operational envelope based at least in part on operator input.

The method may involve generating a manipulator operational envelope defining boundaries to movement of the distal end of the manipulator within a portion of the instrument workspace, and generating an alert signal when the orientation input signals represent an orientation in the instrument workspace that would cause the distal end of the manipulator or the operational tool to lie on or outside the manipulator operational envelope.

The articulating instrument may include one of a set of articulating instruments of differing instrument types used in a surgical procedure and the manipulator operational envelope may include at least one of one of a pre-defined manipulator operational envelope for all differing instrument types, a pre-defined manipulator operational envelope selected based on the instrument type, a manipulator operational envelope based at least in part on surgical data associated with a target operational site, a manipulator operational envelope based at least in part on surgical data provided by a scan of the target operational site, or a manipulator operational envelope based at least in part on operator input.

The method may involve, in response to receiving position input signals that are associated with a retraction of the distal segment from the instrument workspace, causing the processor circuit to determine whether the position input signal represents a physical position of the distal segment associated with the positioner being in a bent condition, and causing the processor circuit to generate modified position control signals to cause the positioner to be straightened while being retracted into the insertion/retraction region of the instrument workspace.

The method may involve causing the processor circuit to determine whether the received position input signals represent a physical position of the distal segment that lies on or outside the positioner operational envelope and causing the processor circuit to generate the position control signals may involve causing the processor circuit to generate modified position control signals that constrain movements of the distal segment to be within the positioner operational envelope.

The method may involve in response to receiving a positioning mode change signal, causing the processor circuit to combine the position input signals and the orientation input signals to generate a desired position and orientation of the distal end of the manipulator, and perform an inverse kinematic computation on the desired position and orientation of the distal end of the manipulator to determine a position for the distal segment of the positioner and actuation parameters for the positioner and the manipulator associated with the desired position and orientation of the distal end of the manipulator.

Other aspects and features will become apparent to those ordinarily skilled in the art upon review of the following description of specific disclosed embodiments in conjunction with the accompanying figures.

### BRIEF DESCRIPTION OF THE DRAWINGS

In drawings which illustrate disclosed embodiments,

FIG. 1 is a perspective view of a robotic surgery system in accordance with one disclosed embodiment;

FIG. 2A is a front perspective view of a drive unit of the system shown in FIG. 1;

FIG. 2B is a rear perspective view of the drive unit of the system shown in FIG. 1;

FIG. 3A is a perspective view of a portion of an insertion tube of the system shown in FIG. 1;

FIG. 3B is a perspective view of the insertion tube of FIG. 3A including a pair of inserted instruments;

FIG. 3C is a perspective view of an articulated portion of one of the instruments shown in FIG. 3B;

FIG. 4 is a block diagram of processor circuit elements of the robotic surgery system shown in FIG. 1;

FIG. 5 is a perspective view of a portion of an input device of the system shown in FIG. 1;

FIG. 6 is a process flowchart depicting blocks of code for directing the processor circuit of FIG. 4 to receive input signals from the input device of FIG. 5;

FIG. 7A is a rear perspective view the articulated portions of the instruments shown in FIG. 3B;

FIG. 7B is a side perspective view of one of the instruments shown in FIG. 3B;

FIG. 8 is a perspective view of an alternative input device used to generate position input signals and orientation input signals for use in the system shown in FIG. 1;

FIG. 9 is a perspective view of an alternative embodiment of an instrument;

FIG. 10 is a front perspective view of the insertion tube of FIG. 3A including an inserted instrument;

FIG. 11A is an elevational view of the insertion tube and inserted instrument in a first condition;

FIG. 11B is an elevational view of the insertion tube and inserted instrument in a second condition;

FIG. 11C is an elevational view of the insertion tube and inserted instrument in a third condition;

FIG. 11D is an elevational view of the insertion tube and instrument in a bent condition;

FIG. 12 is a process flowchart including block of code for directing the processor circuit of FIG. 4 to generate a positioner operational envelope;

FIG. 13A is a perspective view of the articulated portion of the instrument shown in FIG. 3C in a pose in accordance with disclosed embodiments; and

FIG. 13B is a perspective view of the articulated portion of the instrument shown in FIG. 3C in a pose in accordance with a prior art embodiment.

### DETAILED DESCRIPTION

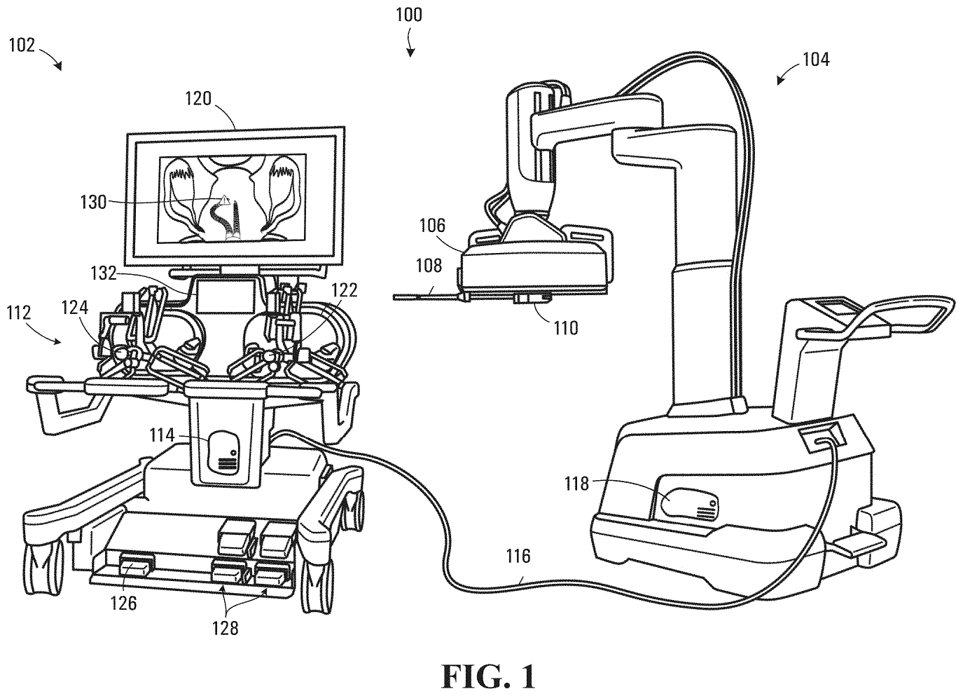

Referring to FIG. 1, a robotic surgery system in accordance with one disclosed embodiment is shown generally at 100. The system 100 includes a workstation 102 and an instrument cart 104. The instrument cart 104 includes a drive unit 106 to which an insertion tube 108 and an instrument 110 are mounted. The workstation 102 includes an input device 112 that receives operator input and produces input signals. The input device 112 may also be capable of generating haptic feedback to the operator. The input device 112 may be implemented using a haptic interface available from Force Dimension, of Switzerland, for example. However in other embodiments the input device may be implemented using other input devices, including but not limited to a non-contact hand tacking device or other motion sensing device.

In the embodiment shown, the workstation 102 further includes a workstation processor circuit 114 in communication with the input device 112 for receiving the input signals and generating control signals for controlling the robotic surgery system, which are transmitted to the instrument cart 104 via an interface cable 116. In this embodiment, the input device 112 includes right and left hand controllers 122 and 124, which are grasped by the operator's hands and moved to cause the input device 112 to produce the input signals. The workstation 102 also includes a footswitch 126 for generating an enablement signal. The workstation 102 may also include other footswitches 128 that provide an additional input to the system as described below. The workstation 102 also includes a display 120 in communication with the workstation processor circuit 114. The display 120 may be configured for displaying images of a surgical site and/or portions of the instrument 110 in the surgical site. In the embodiment shown, the workstation 102 further includes a secondary display 132 for displaying status information related to the system 100. The instrument cart 104 includes an instrument processor circuit 118 that receives the input signals from the workstation processor circuit 114 and produces control signals for causing movement of the instrument 110 during a surgical procedure.

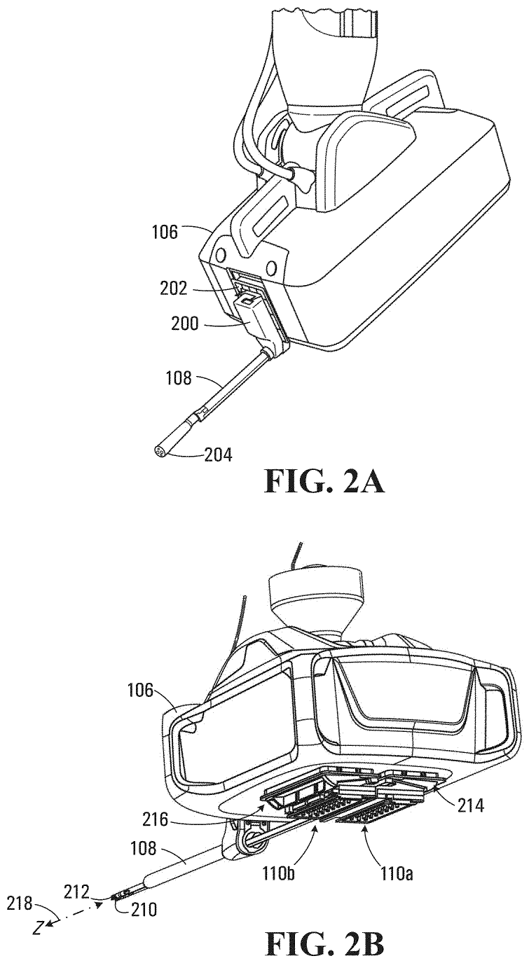

The drive unit 106 is shown in isolation in FIGS. 2A and 2B. Referring to FIG. 2A, the insertion tube 108 includes a drive interface 200 that detachably mounts to a corresponding drive interface 202 on the drive unit 106. The insertion tube 108 includes a camera 204 at a distal end of the insertion tube, which is inserted into a body cavity of a patient to capture body cavity image data representing an interior view of the body cavity for display on the display 120 of the workstation 102. Referring to FIG. 2B, in this embodiment the insertion tube 108 includes a pair of adjacent bores extending through the insertion tube for receiving a right hand side instrument 110a and a left hand side instrument 110b. The instruments 110a and 110b each include a respective operational tool 210 and 212 at a distal end. The operational tools 210 and 210 may be one of a variety of different operational tools, such as a probe, dissector, hook, or cauterizing tool. As an example, the operational tools 210 and 210 may be configured as an end effector having opposing jaws that provide an actuated function such as a scissor for cutting tissue or forceps for gripping tissue. In other embodiments one of the instruments 110a or 110b may include an operational tool 210 or 212 in the form of a distally located camera that provides imaging functions in addition to or in place of the camera 204. One of the instruments 110a or 110b may include an operational tool in the form of an illuminator configured to provide illumination for generation of images by the camera 204.

The drive unit 106 includes a mounting interface 214 for mounting the instrument 110a and a mounting interface 216 for mounting the instrument 110b. The drive unit 106 is configured to cause the respective mounting interfaces 214 and 216 to advance or retract in a direction aligned with a Z-axis shown at 218.

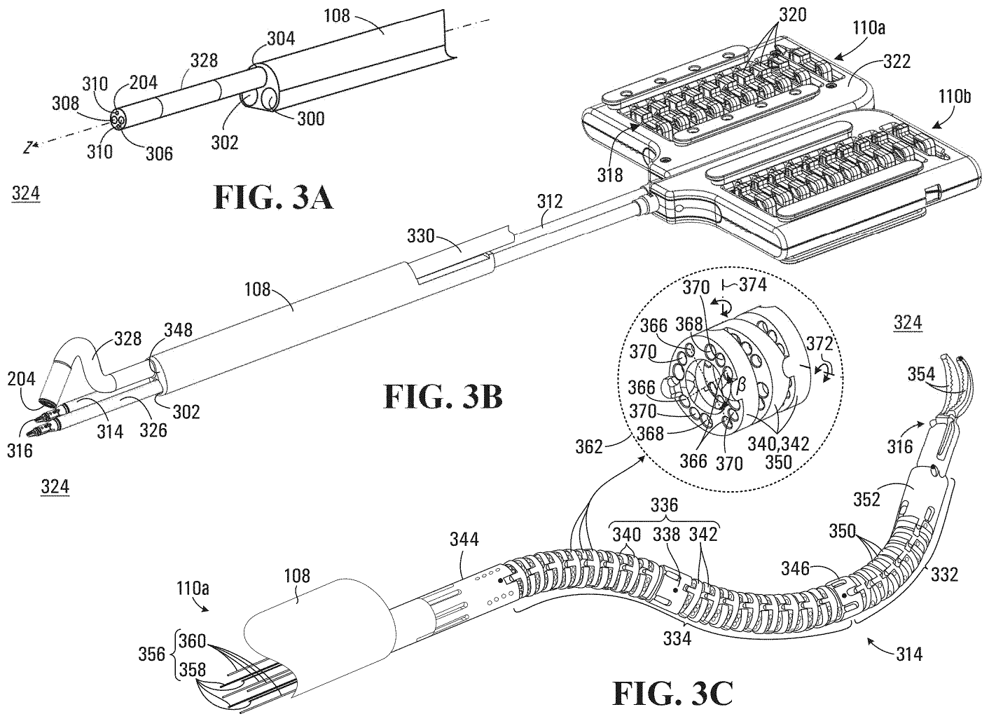

A portion of the insertion tube 108 is shown in FIG. 3A and includes two adjacently located bores 300 and 302 extending through the insertion tube 108 for receiving the respective surgical instruments 110a and 110b. The insertion tube 108 may be inserted through an incision into a body cavity of a patient to provide access to an instrument workspace 324 within a surgical site. The insertion tube 108 also includes a third bore 304 for receiving the camera 204. In other embodiments the insertion tube 108 may include additional bores for accommodating further instruments.

The camera 204 is configured as a stereoscopic camera having a pair of spaced apart imagers 306 and 308 for producing stereoscopic views representing an interior view of the body cavity. The camera 204 also includes an integrated illuminator 310 for illuminating the body cavity for capturing images. The integrated illuminator 310 may be implemented using an illumination source such as a light emitting diode, or an illumination source may be remotely located and may deliver the illumination through an optical fiber running through the insertion tube 108.

The camera 204 is mounted on an articulated arm 328 and coupled to a flexible shaft 330, which is shown truncated in FIG. 3B. The flexible shaft 330 will typically include a connector end that is connected to a camera port in the drive unit 106. The camera 204 is shown in a deployed state in FIG. 3B. Drive forces delivered by the drive unit 106 via the drive interface 202 to the drive interface 200 of the insertion tube 108 cause the articulated arm 328 to move the camera 204 the longitudinally extended insertion state shown in FIG. 3A to a deployed state as shown in FIG. 3B. In the deployed position, the camera 204 is able to generate images of the surgical site and instrument workspace 324 without obstructing movements of the instruments 110a and 110b. The images of the surgical site may be displayed on the display 120 of the system 100 shown in FIG. 1.

The instruments 110a and 110b are shown inserted through the respective bores 300 and 302 of the insertion tube 108 (in FIG. 3B the bore 302 is not visible). The right hand side instrument 110a includes a rigid shaft portion 312 and an articulated portion 314 that extends outwardly from the bore 300. In this embodiment the operational tool 210 of the instrument 110a is an end effector 316. The instrument 110a includes an actuator 318, which includes a plurality of actuator slides 320 disposed in an actuator housing 322. The actuator housing 322 is located at a proximal end of the instrument 110a that couples to the mounting interface 214 on the drive unit 106 for moving the articulated portion 314 and actuating the end effector 316. The rigid shaft portion 312 may be advanced or retracted by the mounting interface 214 to change the insertion depth of the articulated portion 314 within the instrument workspace 324. The instrument workspace 324 extends outwardly from an end 348 of the insertion tube 108.

The actuator 318 of the instrument 110a may be generally configured as disclosed in commonly owned PCT patent publication WO2016/090459 entitled “ACTUATOR AND DRIVE FOR MANIPULATING A TOOL” filed on Feb. 18, 2015 and incorporated herein by reference in its entirety. The interface of the drive unit 106 may have a track system (not shown) coupled to the actuator 318 for longitudinally advancing and retracting the instrument 110a to cause the rigid shaft portion 312 to move within the bore 300. The longitudinal positioning of the instrument 110a places the end effector 316 at a desired longitudinal offset with respect to the insertion tube 108 for accessing a surgical site within the body cavity of the patient.

The instrument 110b is shown in FIG. 3B in side-by side relation and identically configured to the instrument 110a and includes an articulated portion 326 extending into the surgical site. In some embodiments, the instrument 110b may have a different operational tool than the instrument 110a.

The articulated portion 314 of the instrument 110a is shown actuated and in enlarged detail in FIG. 3C. Referring to FIG. 3C, the articulated portion 314 of the instrument includes a manipulator 332 and a positioner 334. The positioner 334 is actuable to position a distal segment 346 of the positioner within an instrument workspace 324. The manipulator 332 is attached to the distal segment 346 of the positioner 334 and includes a distal end 352. The distal end 352 is configured for mounting the end effector 316 for performing an operation within the instrument workspace 324. In this embodiment, the end effector 316 includes a pair of jaws 354 for grasping tissue. The manipulator 332 is actuable to manipulate the distal end 352 of the manipulator within the instrument workspace 324.

The positioner 334 includes a first plurality of segments 336 extending between a bulkhead segment 344 and the distal segment 346. In the embodiment shown the first plurality of segments 336 include the bulkhead segment 344, a set of fifteen stacked vertebra segments 340 extending between the bulkhead segment and an intermediate segment 338. A further set of fifteen stacked vertebra segments 340 extend between the intermediate segment 338 and the distal segment 346. Three adjacent vertebra 340 are shown in enlarged detail in an insert 362. The adjacent vertebra 340 are coupled to move about either a pitch axis 372 or a yaw axis 374 with respect to the adjacent vertebra, which facilitates bending of the positioner 334 in a continuous arc.

The manipulator 332 includes a second plurality of segments 350 extending between the distal segment 346 of the positioner 334 and the distal end 352 of the manipulator. Each manipulator segment 350 may be similarly configured to the segments shown in the insert 362.

A plurality of control wires 356 are shown in FIG. 3C extending into the articulated portion 314 of the instrument 110a. Although not visible in FIG. 3B, the plurality of control wires 356 extend back along the rigid shaft portion 312 and each control wire is coupled to one of the plurality of actuator slides 320 within the actuator housing 322. The control wires 356 may each be implemented as a single flexible wire, which in one embodiment may be implemented using nitinol wire, which is capable of about 200N in tension or compression without permanent deformation and capable of experiencing up to about 4% strain. Nitinol is an alloy of nickel and titanium having shape memory and superelasticity and its ability to support both tension and compression allows the control wires to be selectively pushed or pulled with similar forces without permanent deformation.

The plurality of control wires 356 include four positioner control wires 358 extending through the insertion tube 108, through a plurality of openings 366 in the first plurality of segments 336, and are connected to the distal segment 346. A first pair of the positioner control wires 358 are routed through a first pair of the openings 366 that are diametrically opposed, as shown in the insert 362. The remaining pair of positioner control wires 358 are routed through a second pair of the openings 366 that are orthogonally located with respect to the first pair of openings. The articulated portion 314 of the instrument 110a may be configured as described in further detail in commonly owned PCT patent publication WO2014/201538 entitled “ARTICULATED TOOL POSITIONER AND SYSTEM EMPLOYING SAME” filed on Dec. 20, 2013 and incorporated herein by reference in its entirety. The described positioner provides for dexterous movement of the end effector 316 through a plurality of articulated segments. As described in more detail in PCT patent publication WO2014/201538, the control wires may be configured to work in pairs connected to diametrically opposed portions of the distal segment 346. When a pushing force is delivered to one of the pair of control wires, a pulling force is delivered to the other of the pair. In other embodiments, the actuation may be provided by a single wire that is pulled or pushed to cause the desired movement of the distal segment 346. Selectively actuating the first plurality of control wires 358 cause the respective stacked vertebra 340 and 342 to move to position the distal segment at the physical position represented by the position input signal while the respective vertebra are angled with respect to each other to bend the instrument in a continuous arc.

The plurality of control wires 356 also include four manipulator control wires 360 extending through the insertion tube 108, through a plurality of openings 368 in the segments 336, through a plurality of openings in the second plurality of segments 350, and connected to the distal end 352 of the manipulator 332. As in the case of the positioner control wires 358, the four manipulator control wires 360 are routed through openings 368 indicated in the insert 362. When the four manipulator control wires 360 are actuated by the respective actuator slides 320, an actuation force is applied to the distal end 352. The manipulator 332 is thus actuable via the plurality of actuator slides 320 to manipulate the distal end 352 and the end effector 316 within the instrument workspace 324.

In the embodiment shown in FIG. 3C, the segments 336 in the positioner 334 each include four openings 370 that are additional to the four openings required for the four positioner control wires 358 and the four openings required for the four manipulator control wires 360. These four openings 370 are respectively located between the openings 366 and the openings 368, as shown in the insert 362. The additional openings 370 accommodate four structural wires (not shown) that are connected at the bulkhead segment 344 and run through the openings in the second plurality of segments 350 to the distal segment 346, where the structural wires are connected to the distal segment. The structural wires each have the same length and function as a parallelogram in two dimensions, tending to keep the distal segment 346 in the same orientation as the bulkhead segment 344. When pushing and pulling on the pairs of positioner control wires 358, the equal length structural wires constrain the set of segments 340 to bend in one direction, while the set of segments 342 bend in an opposite direction, thus causing the positioner 334 to take up an “S” shape as shown in FIG. 3C. The pushing and pulling on the pairs of positioner control wires 358 thus cause the distal segment 346 to move laterally and/or vertically, while remaining substantially aligned with the bulkhead segment 344 and the Zo-axis. The structural wires need not necessarily be secured within the bulkhead segment 344. In some embodiments the structural wires may be secured within the rigid shaft portion 312 or actuator housing 322.

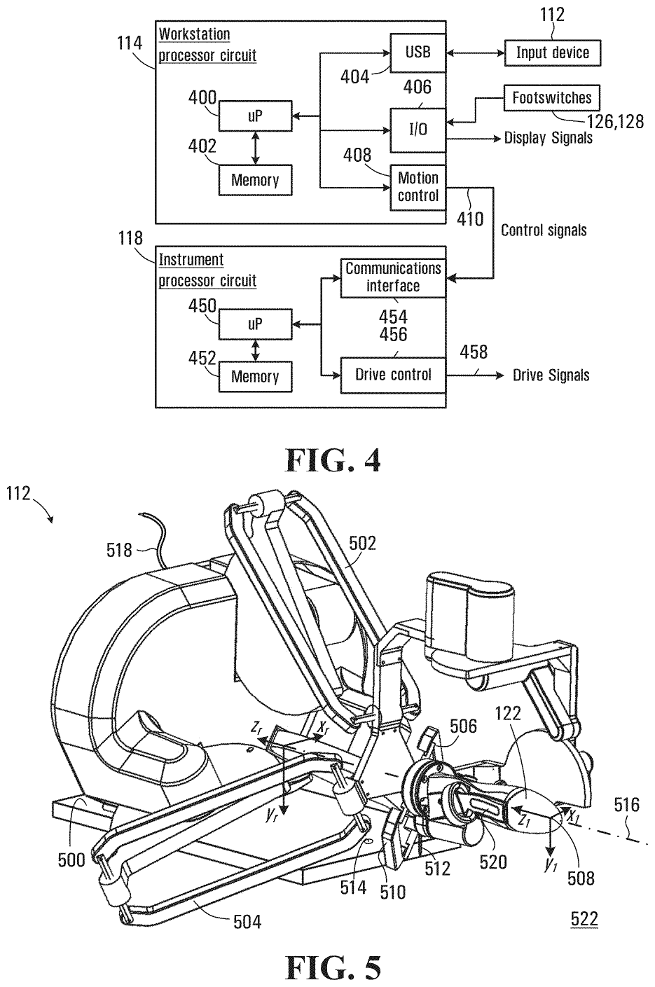

A block diagram of processor circuit elements of the robotic surgery system 100 is shown in FIG. 4. Referring to FIG. 4 the workstation processor circuit 114 includes a microprocessor 400, a workstation memory 402, a USB interface 404, an input/output 406, and a motion control interface 408, all of which are in communication with the microprocessor 400. In this embodiment the input device 112 communicates using a USB protocol and the USB interface 404 receives input signals produced by the input device in response to movements of the hand controllers 122 and 124. The input/output 406 includes an input for receiving the enablement signal from the footswitches 126 and 128 and an output for producing display signals for driving the display 120 (shown in FIG. 1). The motion control interface 408 generates control signals 410 based on the input signals received from the input device 112.

The instrument processor circuit 118 includes a microprocessor 450, a memory 452, a communications interface 454, and a drive control interface 456, all of which are in communication with the microprocessor. The microprocessor 450 receives the control signals 410 at the communications interface 454 via the interface cable 116 (FIG. 1). The microprocessor 450 processes the control signals 410 and causes the drive control interface 456 to produce drive signals 458 for moving the instruments 110a and 110b. The drive signals are received by the drive unit 106, which generates the necessary actuation forces for moving the plurality of actuator slides 320 to position the positioner 334 and the manipulator 332 within the instrument workspace 324.

The workstation processor circuit 114 thus acts as a controller subsystem for receiving user input, while the instrument processor circuit 118 acts as a responder subsystem in responding to the control signals 410 based on user input by driving the instruments 110a and 110b. While the embodiment shown includes both the workstation processor circuit 114 and the instrument processor circuit 118, in other embodiments a single processor circuit may be used to perform both controller and responder functions.

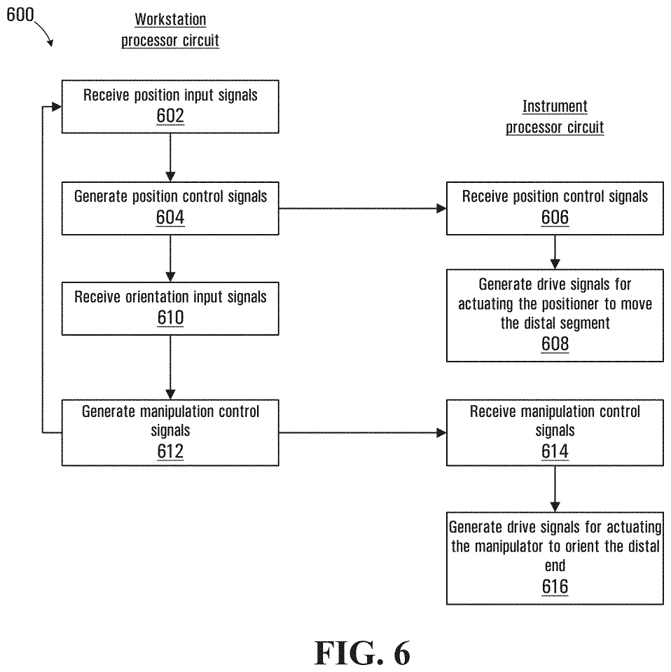

A portion of the input device 112 that includes the right hand controller 122 is shown in greater detail in FIG. 5. For simplicity, only the right hand controller 122 of the input device 112 will be further described, it being understood that the left hand controller 124 operates in the same way. The input device 112 is supported on a base 500 and includes arms 502, 504, and 506 that provide a mounting for the hand controller 122, which may be grasped by the operator and moved within an input workspace. The input device reference frame has an xᵣ-zᵣ plane parallel to the base 500 and a yᵣ axis perpendicular to the base. The zᵣ axis is parallel to the base 500 and is coincident with an axis 516 passing centrally through the hand controller 122. The xᵣ, yᵣ, zᵣ reference frame defines an input workspace 522.

The input device 112 includes a plurality of encoders (not shown) that generate signals in response to movements of the movements of the arms 502, 504, and 506, which act as a via a first kinematic structure for kinematically transmitting translational movements of the operator's hand via the hand controller 122 in three translational degrees of freedom to the encoders. The input device 112 is thus operable to produce translational movement signals based on the encoder signals to generate the position input signals. The arms 502-506 permit positioning and rotation about orthogonal axes xᵢ, yᵢ, and zᵢ of a Cartesian reference frame. The Cartesian reference frame has an origin at a point on a body of the hand controller 122 and the location of the origin defines the hand controller position 508. In this embodiment, the hand controller 122 is mounted on a gimbal mount 510. The arms 502-506 confine movements of the hand controller 122 and hence the hand controller position 508 to within a generally hemispherical input workspace.

The input device 112 includes a second kinematic structure that kinematically transmits orientation movements of the operator's hand in the at least two rotational degree of freedom to encoders (not shown) that sense the rotational orientation of the hand controller 122 about xᵢ, and yᵢ axes. In this embodiment the input device 112 is also responsive to rotations of the hand controller 122 about the zᵢ-axis, for a third degree of freedom. Rotational hand movements of the operator are thus also encoded to produce signals representing the orientation of the hand controller 122 in the input workspace 522 relative to an input device Cartesian reference frame xᵣ, yᵣ, zᵣ. The encoder orientation signals are processed by the input device 112 to generate the orientation input signals.

In one embodiment the input device 112 may also be configured to generate haptic forces for providing haptic feedback to the hand controller 122 through the arms 502-506 and gimbal mount 510. For example, haptic forces may be initiated in response to alert signals that are generated by the workstation processor circuit 114 or instrument processor circuit 118 and communicated to the input device 112. The hand controller 122 also includes an end effector actuator 520 that may be opened and closed to actuate movement of an end effector as described in more detail later herein.

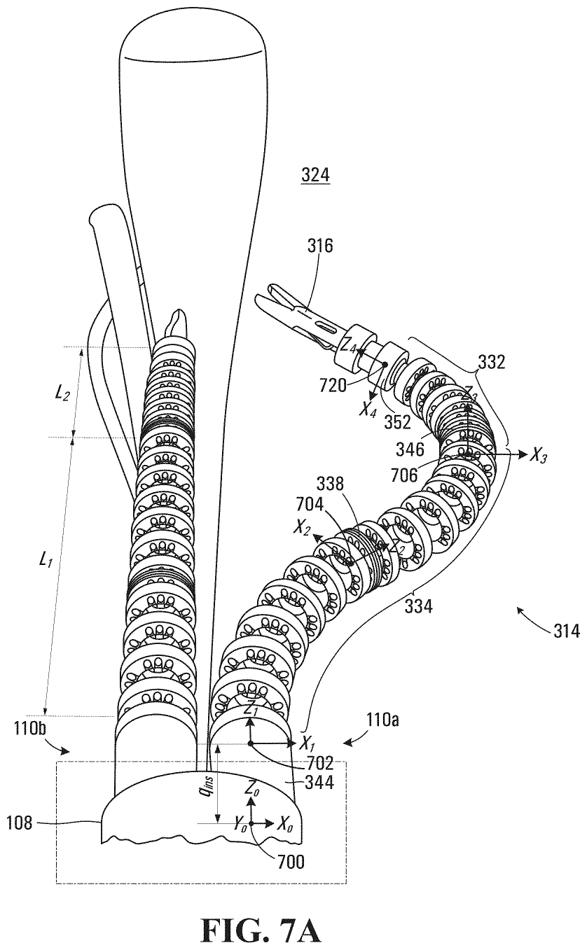

Referring to FIG. 6, a flowchart depicting blocks of code for directing the workstation processor circuit 114 to receive input signals from the input device 112 for controlling the articulated portion 314 of the instrument 110a is shown at 600. The blocks generally represent codes that may be read from the workstation memory 402 for directing the microprocessor 400 to perform control functions. The actual code to implement each block may be written in any suitable program language, such as C, C++, C #, Java, and/or assembly code, for example.

The process 600 is initiated at block 602, which directs the microprocessor 400 to receive position input signals generated by the input device 112 in response to movements of the hand controller 122. For the input device 112 shown in FIG. 5, the input workspace 522 is represented by an input device reference frame xᵣ, yᵣ, zᵣ. The position input signals are generated by the input device 112 in response to translations of the hand controller 122 in three translational degrees of freedom within the input workspace by the operator. In this embodiment, the position and orientation signals are transmitted as input signals via the USB connection 518 to the USB interface 404 of the workstation processor circuit 114. The received position input signal represents a current position of the hand controller 122 within the reference frame xᵣ, yᵣ, zᵣ, and may be represented by a position vector given by:

$${\vec{P}}_{MCURR} = \{ \begin{matrix}{x}_{i} \\ {y}_{i} \\ {z}_{i}\end{matrix} \} , \qquad Eqn 1$$

where xᵢ, yᵢ, and zᵢ represent coordinates of the hand controller position 508 (i.e. the origin of the coordinate system xᵢ, yᵢ, zᵢ) relative to the input device reference frame xᵣ, yᵣ, zᵣ.

Block 604 then directs the microprocessor 400 to generate position control signals based on the received position input signals and to transmit the position control signals to the instrument processor circuit 118 via the motion control interface 408.

The process then continues at block 606, which is implemented on the instrument processor circuit 118. Block 606 directs the microprocessor 450 to receive the position control signals. Block 608 then directs the microprocessor 450 to generate drive signals. The drive control interface 456 of the instrument processor circuit 118 produces the necessary drive signals to cause the drive unit 106 to actuate the applicable plurality of actuator slides 320 on the instrument 110a for actuating the positioner 334 to move the distal segment 346 to a physical position represented by the position input signals.

Block 610 then directs the microprocessor 400 of the workstation processor circuit 114 to receive the orientation input signals from the input device 112 at the USB interface 404. The orientation of the hand controllers 122 within the input workspace 522 is given by a rotation matrix:

$${R}_{MCURR} = [ \begin{matrix}{x}_{1 x} & {y}_{1 x} & {z}_{1 x} \\ {x}_{1 y} & {y}_{1 y} & {z}_{1 y} \\ {x}_{1 z} & {y}_{1 z} & {z}_{1 z}\end{matrix} ] , \qquad Eqn 2$$

where the columns of the matrix represent the axes of the hand controller reference frame xᵢ, yᵢ, zᵢ relative to the input device reference frame xᵣ, yᵣ, zᵣ. The matrix R_{MCURR} thus defines the current rotational orientation of the hand controller 122 with the input workspace 522 relative to the xᵣ, yᵣ, and zᵣ reference frame. The current hand controller position vector \vec{P}_{MCURR} and current handle rotation matrix R_{MCURR} are received as current hand controller position signals and current hand controller orientation signals via the USB connection 518 at the USB interface 404 of the workstation processor circuit 114.

Block 612 then directs the microprocessor 400 to processes the position input signals and to generate position control signals for actuating the positioner to move the distal segment 346 within the instrument workspace 324. The position control signals are transmitted via the motion control interface 408 to the instrument processor circuit 118.

The process then continues at block 614, which directs the microprocessor 450 of the instrument processor circuit 118 to receive the manipulation control signals. Block 616 then directs the microprocessor 450 to generate drive signals to cause the drive unit 106 to actuate the applicable plurality of actuator slides 320 on the instrument 110a for actuating the manipulator 332 to orient the distal end 352 within the instrument workspace 324.

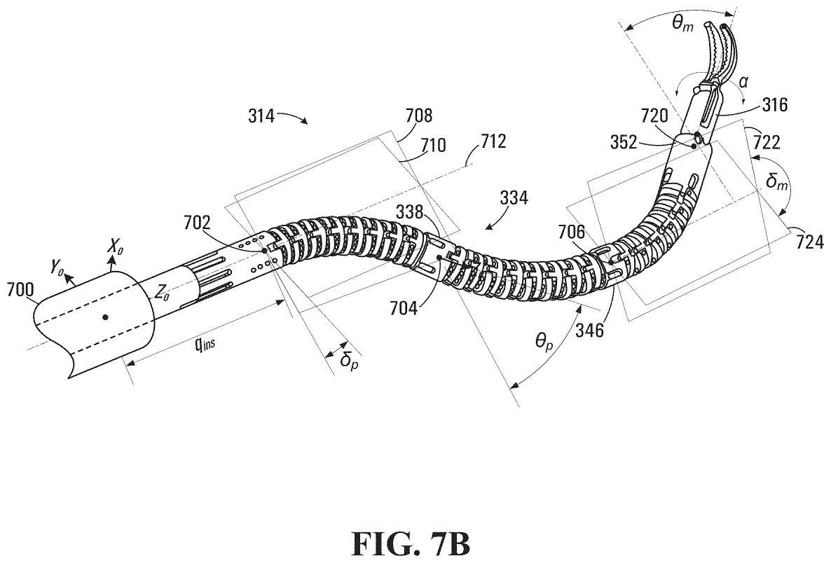

The articulated portion 314 of the instrument 110a and an articulated portion of the instrument 110b are shown from the rear in FIG. 7A. The articulated portion 314 of the instrument 110a is also shown from a side perspective in FIG. 7B. Referring to FIG. 7A, a fixed reference frame 700 (X₀, Y₀, Z₀) is established for the articulated portion 314 of the instrument 110a. In this embodiment the fixed reference frame 700 has an origin located at a center of the insertion tube 108. In FIG. 7, the Zo axis is aligned with a longitudinal axis of the insertion tube 108 and the Y-axis is directed into the plane of the page.

The bulkhead segment 344 has a reference frame 702 (X₁-Z₁) that has an origin located at a center of the bulkhead segment 344. As noted above, the drive unit 106 may cause the actuator housing 322 to be advanced or retracted in the Z₀ axis direction. An insertion distance qᵢₙₛ represents the depth of insertion of the bulkhead segment 344 into the instrument workspace 324 and corresponds to a distance between the origin of the X₀-Z₀ reference frame 700 and the origin of the X₁-Z₁ reference frame 702. The intermediate segment 338 has a reference frame 704 (X₂-Z₂) that has an origin located at the center of the intermediate segment. The distal segment 346 of the positioner 334 has a reference frame 706 (X₃-Z₃) that has an origin located at a center of the distal segment 346.

The position control signal received at block 606 of the process 600 provides the desired location of the reference frame X₃-Z₃ within the instrument workspace 324 with respect to the fixed reference frame X₀-Z₀. Generating the necessary drive signals at block 608 to cause the distal segment 346 to be positioned at X₃Z₃ involves working backwards from the location X₃-Z₃ to determine configuration variables (qᵢₙₛ and the angles θₚ and δₚ) for the positioner 334 and then determining the respective actuations for the positioner control wires 358 that correspond to the configuration variables. The angles θₚ and δₚ are depicted in FIG. 7B. When the positioner 334 is actuated it will bend to lie in a plane 708 that is at an angle δₚ relative to the X₁, Z₁ plane 710. When δₚ=0, the positioner 334 lies on the plane 710. The angle δₚ becomes positive as the positioner rotates away from the Y₁ axis. The angle δₚ represents the degree of bending of the positioner 334 within the plane 708. When the positioner 334 lies in the plane 708 along a line 712 aligned with the Z₀ axis, the angle θₚ=90°. The angle θₚ decreases as the positioner bends outwardly.

A vector p̅_{3/0} from the origin of the fixed reference frame 700 (X₀, Y₀, Z₀) to the end of the positioner 334 (reference frame 706), has x, y, and z components written in the fixed reference frame as:

$${\overline{p}}_{3 / 0} \cdot \overline{ι} = \frac{- {L}_{1} cos {δ}_{p} ( sin {θ}_{p} - 1 )}{\frac{π}{2} - {θ}_{p}} \qquad Eqn 3 a$$

$${\overline{p}}_{3 / 0} \cdot \overline{J} = \frac{{L}_{1} sin {δ}_{p} ( sin {θ}_{p} - 1 )}{\frac{π}{2} - {θ}_{p}} \qquad Eqn 3 b$$

$${\overline{p}}_{3 / 0} \cdot \overline{k} = {q}_{i n s} + \frac{{L}_{1} cos {θ}_{p}}{\frac{π}{2} - {θ}_{p}} \qquad Eqn 3 c$$

where:
ī, j̅, k̅ are unit vectors in the x, y, and z directions, respectively; and
L₁ is the length of the positioner 334 (shown in FIG. 7A for the instrument 110b).

To find the angles δₚ and θₚ of the proximal segment (s-segment) the equations (1a) and (1b) must be solved for δₚ and θₚ. δₚ can be found directly by taking the ratio of (3b) and (3a):

δₚ=atan 2(−p̅_{3/0·}ĵ,p̅_{3/0·}î) Eqn 4

To find θₚ, (1a) or (1b) must be solved for θₚ. A closed-form solution does not exist, so the solution must be found numerically. The Newton-Raphson method has been used successfully in simulation. This method is appropriate and tends to converge very quickly because in the range 0≤θₚ≤π the function (13) has a large radius of curvature. The Newton-Raphson method may be implemented using equations (1a) and (1b), which can be rearranged to be written in the form ƒ(θₚ)=0:

$$f ( {θ}_{p} ) = \frac{- {L}_{1}}{\frac{π}{2} - {θ}_{p}} \cdot cos {δ}_{p} \cdot ( sin {θ}_{p} - 1 ) - {\overline{p}}_{3 / 0} \cdot \hat{ι} + \frac{{L}_{1}}{\frac{π}{2} - {θ}_{p}} \cdot sin {δ}_{p} \cdot ( sin {θ}_{p} - 1 ) - {\overline{p}}_{3 / 0} \cdot \hat{} = 0 \qquad Eqn 5$$

Note that the inclusion of both equations (3a) and (3b) is necessary, since either one individually is stationary in θₚ for specific values of δₚ. For example, for

$${δ}_{p} = \frac{π}{2} ,$$

equation (3a) is equal to zero for all values of θₚ. The derivative of Eqn 5 with respect to θₚ is:

$${f}^{′} ( {θ}_{p} ) = \frac{{L}_{P}}{\frac{π}{2} - {θ}_{P}} \cdot [ \frac{- 1}{\frac{π}{2} - {θ}_{P}} \cdot cos {δ}_{p} \cdot ( sin {θ}_{p} - 1 ) - cos {δ}_{p} \cdot cos {θ}_{p} + \frac{1}{\frac{π}{2} - {θ}_{P}} \cdot sin {δ}_{p} \cdot ( sin {θ}_{p} - 1 ) + {δ}_{p} \cdot cos {θ}_{p} ] \qquad Eqn 6$$

Following the Newton-Raphson scheme, successive iterations can be made for improved estimates of θₚ to satisfy (13) using the following relationship:

$${θ}_{{p}_{n + 1}} = {θ}_{{p}_{n}} - \frac{f ( {θ}_{{p}_{n}} )}{{f}^{′} ( {θ}_{{p}_{n}} )} \qquad Eqn 7$$

The choice of an initial θₚ can determine the number of total iterations required in order to reach a desired amount of error. It is possible that the required number of iterations can be minimized by setting the initial θₚ equal to the θₚ value from the previous kinematics frame. This, however, can increase software complexity and creates special cases that must be handled, such as the case with

$${θ}_{p} = \frac{π}{2} .$$

In order to find qᵢₙₛ, equation (3c) can be rearranged as follows:

$${q}_{ins} = {\overline{p}}_{3 / 0} . \overline{k} - \frac{{L}_{1} cos {θ}_{p}}{\frac{π}{2} - {θ}_{p}} \qquad Eqn 8$$

The respective actuations for the positioner control wires 358 p₁, p₂, p₃, and p₄ may be calculated from the calculated angles θₚ and δₚ:

$${p}_{1} = {r}_{b} ( {θ}_{p} - \frac{π}{2} ) cos ( {δ}_{p} ) \qquad Eqn 9 a$$

$${p}_{2} = {r}_{b} ( {θ}_{p} - \frac{π}{2} ) cos ( {δ}_{p} - β ) \qquad Eqn 9 b$$

$${p}_{3} = {r}_{b} ( {θ}_{p} - \frac{π}{2} ) cos ( {δ}_{p} - 2 β ) \qquad Eqn 9 c$$

$${p}_{4} = {r}_{b} ( {θ}_{p} - \frac{π}{2} ) cos ( {δ}_{p} - 3 β ) , \qquad Eqn 9 d$$

where r_{b} is the radius of the segments 336 and ß is the angle between the openings in the segments 336 (in this case ß=90°). The wires p₁ and p₃ correspond to the first pair of control wires and the wires p₂ and p₄ correspond to the second pair of control wires. The above equations may thus be evaluated by the microprocessor 450 of the instrument processor circuit 118 to determine actuations p₁, p₂, p₃, and p₄ for causing the positioner 334 to position the distal segment 346 at a physical position in the instrument workspace 324 corresponding to the position control signals received from the workstation processor circuit 114 at block 606.

Still referring to FIG. 7A, the distal segment 346 is thus positioned in the instrument workspace 324 based on the position control signals. The manipulator 332 extends into the instrument workspace 324 from the distal segment 346 and is capable of movement in at least two degrees of freedom with respect to the distal segment. The distal end 352 of the manipulator 332 has a reference frame 720 (X₄-Z₄) that has an origin located at a center of the distal end. The movements of the distal end 352 of the manipulator 332 may be characterized as occurring within a bend plane 722, which is disposed at an angle δₘ to a plane 724 aligned with the X₃-Z₃ axes of the reference frame 706 at the distal segment 346. Within the bend plane 722, the manipulator 332 is disposed at an angle θₘ, with respect to the Z₃-axis.

A vector p̅_{4/3} from the origin of the distal segment 346 reference frame 706 to the distal end 352 of the manipulator 332 (reference frame 720), has x, y, and z components written in the fixed reference frame 700 as:

$${\overline{p}}_{4 / 3} . \overline{ι} = \frac{- {L}_{2} cos {δ}_{m} ( sin ( {θ}_{m} ) - 1 )}{\frac{π}{2} - {θ}_{m}} \qquad Eqn 10 a$$

$${\overline{p}}_{4 / 3} . \overline{} = \frac{{L}_{2} sin {δ}_{m} ( sin ( m ) - 1 )}{\frac{π}{2} - {θ}_{m}} \qquad Eqn 10 b$$

$${\overline{p}}_{4 / 3} . \overline{k} = \frac{{L}_{2} cos ( {θ}_{m} )}{\frac{π}{2} - {θ}_{m}} \qquad Eqn 10 c$$

where:
ī, j̅, k̅ are unit vectors in the x, y, and z directions, respectively; and
L₂ is a length of the manipulator 332, shown in FIG. 7A for the instrument 110b.

The rotation matrix between the end of the positioner 334 (frame X₃-Z₃) and the end of the distal end 352 (frame X₄-Z₄) may be computed as a series of simple rotations including rotation of δₘ about the Z-axis (R_{(δ}_{ₘ}₎), followed by rotation of

$$( \frac{π}{2} - {θ}_{m} )$$

and about the new

$$Y - axis ( {R}_{( \frac{π}{2} - {θ}_{m} )} ) ,$$

followed by rotation of −δₘ about the new Z-axis (R_{(-δ}_{ₘ}₎):

$${R}_{4 / 3} = {R}_{( {δ}_{2} )} {R}_{( \frac{π}{2} - {θ}_{2} )} {R}_{( - {δ}_{2} )} \qquad Eqn 11$$

$${R}_{4 / 3} = [ \begin{matrix}cos ( {δ}_{m} ) & - sin ( {δ}_{m} ) & 0 \\ sin ( {δ}_{m} ) & cos ( {δ}_{m} ) & 0 \\ 0 & 0 & 1\end{matrix} ] [ \begin{matrix}cos ( \frac{π}{2} - {θ}_{m} ) & 0 & sin ( \frac{π}{2} - {θ}_{m} ) \\ 0 & 1 & 0 \\ - sin ( \frac{π}{2} - {θ}_{m} ) & 0 & cos ( \frac{π}{2} - {θ}_{m} )\end{matrix} ] [ \begin{matrix}cos ( - {δ}_{m} ) & - sin ( - {δ}_{m} ) & 0 \\ sin ( - {δ}_{m} ) & cos ( - {δ}_{m} ) & 0 \\ 0 & 0 & 1\end{matrix} ] \\ {R}_{4 / 3} = [ \begin{matrix}\begin{matrix}{sin}^{2} {δ}_{m} + \\ sin {θ}_{m} {cos}^{2} {δ}_{m}\end{matrix} & cos {δ}_{m} sin {δ}_{m} sin {θ}_{m} - cos {δ}_{m} sin {δ}_{m} & \begin{matrix}cos {δ}_{m} \\ cos {θ}_{m}\end{matrix} \\ \begin{matrix}cos {δ}_{m} sin {δ}_{m} \\ sin {θ}_{m} - \\ cos {δ}_{m} sin {δ}_{m}\end{matrix} & \begin{matrix}{cos}^{2} {δ}_{m} + \\ sin {θ}_{m} {sin}^{2} {δ}_{m}\end{matrix} & \begin{matrix}sin {δ}_{m} \\ cos {θ}_{m}\end{matrix} \\ - cos {δ}_{m} cos {θ}_{m} & - sin {δ}_{m} cos {θ}_{m} & sin {θ}_{m}\end{matrix} ]$$

R_{4/3} may be written in short form as:

$${R}_{m} = {R}_{4 / 3} = [ \begin{matrix}{R}_{{m}_{1 , 1}} & {R}_{{m}_{1 , 2}} & {R}_{{m}_{1 , 3}} \\ {R}_{{m}_{2 , 1}} & {R}_{{m}_{2 , 2}} & {R}_{{m}_{2 , 3}} \\ {R}_{{m}_{3 , 1}} & {R}_{{m}_{3 , 2}} & {R}_{{m}_{3 , 3}}\end{matrix} ] \qquad Eqn 12$$

θₘ can be found by solving for θₘ in the following equation:

$${θ}_{m} = \frac{π}{2} - a tan 2 ( \sqrt{{R}_{{m}_{1 , 3}}^{2} + {R}_{{m}_{2 , 3}}^{2}} , {R}_{{m}_{33}} ) \qquad Eqn 13$$

δₘ can be found by taking the ratio:

$$\frac{{R}_{{m}_{2 , 3}}}{{R}_{{m}_{1 , 3}}} = \frac{sin {δ}_{m} cos {θ}_{m}}{cos {δ}_{m} cos {θ}_{m}} = \frac{sin {δ}_{m}}{cos {δ}_{m}} = tan ( {δ}_{m} ) \qquad Eqn 14$$

And then solving for δₘ:

δₘ=atan 2(Rₘ_{_{2,3}},Rₘ_{_{1,3}}) Eqn 15

The respective actuations for the four manipulator control wires 360 m₁, m₂, m₃, and m₄ may be calculated from the calculated angles θₘ and δₘ:

$${m}_{1} = {r}_{b} ( {θ}_{m} - \frac{π}{2} ) cos ( {δ}_{m} - \frac{π}{4} ) \qquad Eqn 16 a$$

$${m}_{2} = {r}_{b} ( {θ}_{m} - \frac{π}{2} ) cos ( {δ}_{m} - \frac{π}{4} - β ) \qquad Eqn 16 b$$

$${m}_{3} = {r}_{b} ( {θ}_{m} - \frac{π}{2} ) cos ( {δ}_{m} - \frac{π}{4} - 2 β ) \qquad Eqn 16 c$$

$${m}_{4} = {r}_{b} ( {θ}_{m} - \frac{π}{2} ) cos ( {δ}_{m} - \frac{π}{4} - 3 β ) \qquad Eqn 16 d$$

where r_{b} is the radius of the segments 336 and ß is the angle between the openings in the segments 336 (in this case ß=90°). The wires m₁ and m₃ correspond to the first pair of control wires and the wires m₂ and m₄ correspond to the second pair of control wires. The above equations may thus be evaluated by the microprocessor 450 of the instrument processor circuit 118 to determine actuations m₁, m₂, m₃, and m₄ for causing the manipulator 332 to orient the distal end 352 at an orientation in instrument workspace 324 corresponding to the orientation control signals received from the workstation processor circuit 114 at block 614.

In this embodiment the end effector 316 connected to the distal end 352 of the manipulator 332 is also configured for an additional roll degree of freedom α about the Z₄-axis in the instrument workspace 324. The roll movement may be actuated in response to a rotation of the hand controller 122 (FIG. 5) about the zᵣ-axis in input workspace 522.

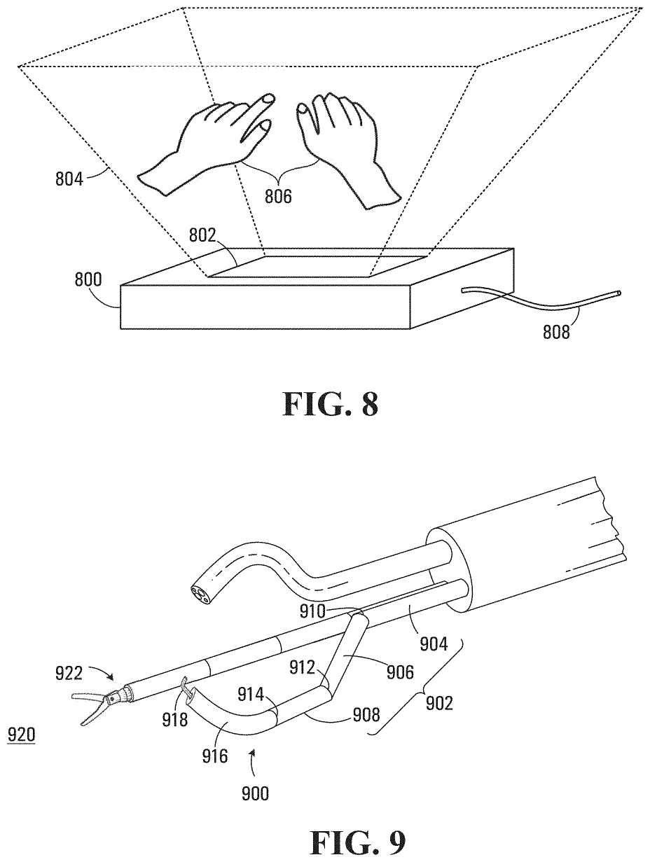

The input device 112 shown in FIG. 1A and FIG. 5 monitors hand movements of the operator via movements of the hand controller 122 grasped by the operator's hand. In another embodiment shown in FIG. 8, an alternative input device may be used to generate the position input signals and orientation input signals. In this embodiment, a motion controller device 800 includes a sensor 802 that monitors free movements of the operator's hands 804 within an input region 806. The workstation processor circuit 114 or another processor circuit within the motion controller device 800 processes signals produced by the sensor 802 to generate position input signals and orientation input signals based on free movements of the operator's hands 804. The input signals may be transmitted via a wired connection to the USB interface 404 or other suitable interface on the workstation processor circuit 114. In some embodiments, the movement signals may be filtered to capture free movements of the operator's hands to extract movements of the operator's digits with respect to either the palm of the operator's hand or the wrist of the operator's hand for generating the input signals. As an example, the motion controller device 800 may be implemented using one of several hand tracking and haptics devices available from Ultraleap of California, United States.

In other embodiments the input signals may be received from other input devices, such as a virtual reality headset worn by the operator. Alternatively, the system 100 may be implemented as an autonomous surgical system that performs surgical operations under the control of an autonomous controller processor circuit. For example, surgical operations performed under the instruction of a surgeon may involve a repetitive step such as performing a suture movement. An autonomous processor circuit may be operably configured to autonomously generate the position input signals and the orientation input signals to perform the suture movement rather than receiving signals from a surgeon via the input device 112.

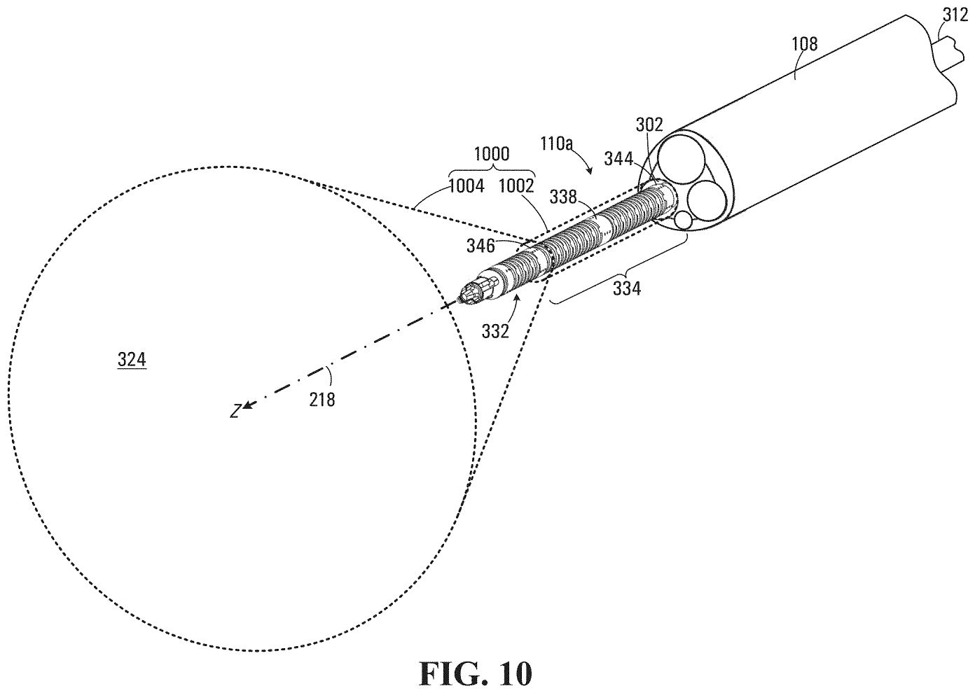

The instrument 110a in the embodiment described above includes articulated segments in the form of vertebra 340, 342, and 350 that provide a smoothly bendable positioner 334. Referring to FIG. 9, in another embodiment an instrument 900 includes a positioner 902 that includes elongated segments 904 and 906, and 908. The segments 904 and 906, and 908 of the positioner 902 are articulated at discrete joints 910, 912, and 914. The articulated segments 904-908 may include control wires (not shown) that run through the linkages and activate the instrument 900 to cause bending at the discrete joints 910-914.

The instrument 900 also includes a manipulable wrist 916, which in this embodiment includes articulated segments as generally described above that provide a smoothly bendable linkage for positioning an end effector 918 in an instrument workspace 920. A second instrument 922 is similarly configured. The above described movement and instrument control embodiments may be implemented for the instruments 900 and 922 with minor implementation differences.

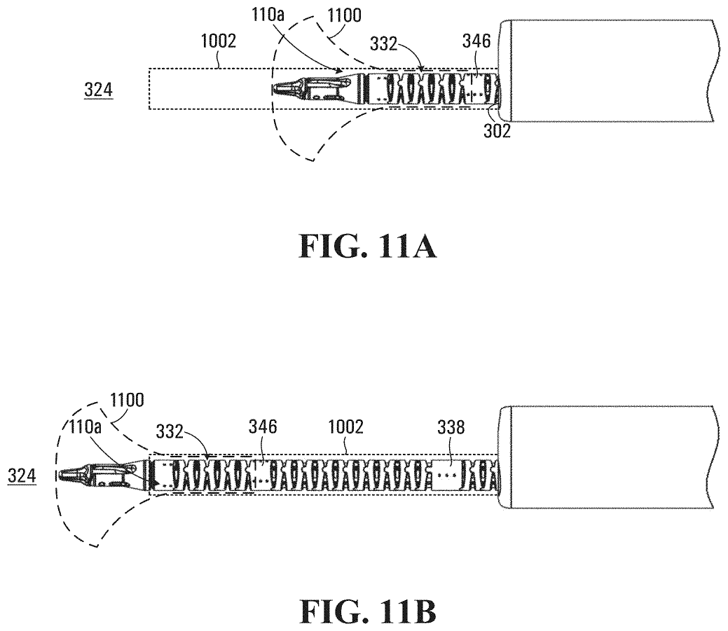

As disclosed above in connection with FIGS. 2A and 2B, the drive unit 106 is configured to cause the respective mounting interfaces 214 and 216 to advance or retract in a direction aligned with a Z-axis shown at 218 causing the positioner 334 and manipulator 332 to either enter the instrument workspace 324 or be withdrawn from the instrument workspace. Referring to FIG. 10, the insertion tube 108 is shown with a single instrument 110a inserted within the bore 302. The instrument 110b and camera 204 have been omitted in FIG. 10. The rigid shaft portion 312 has been advanced or retracted such that the bulkhead segment 344 of the positioner 334 is just clearing the bore 302. If in FIG. 10 the rigid shaft portion 312 is currently being advanced, the positioner 334 is ready for actuation to move the distal segment 346 to a desired physical position within the instrument workspace 324 since the bulkhead segment 344 is clear of the bore 302. However, if the rigid shaft portion 312 is currently being retracted, the positioner 334 would need to have been actuated to assume a straightened condition as shown in FIG. 10, which represents a first constraint on motion of the positioner 334. In the straightened condition the positioner 334 may be retracted into the bore 302 of the insertion tube 108 without causing a collision between the positioner segments and the bore 302. Such collision would work against the actuation of the plurality of actuator slides 320 and may cause wear or damage to the instrument 110a.

If the bulkhead segment 344 is being further advanced out of the bore 302 into the instrument workspace 324, the positioner 334 is able to move the distal segment 346 within movement limitations due to the length of the positioner and the possible bend angles through which the positioner is capable of bending. These physical movement limitations associated with the instrument 110 a place a second constraint on the motion of the positioner 334.

The first and second constraints are illustrated in FIG. 10 as a positioner operational envelope 1000 (shown in broken lines). The positioner operational envelope 1000 includes an insertion/retraction region 1002 associated with the first constraint. The insertion/retraction region 1002 represents a region of the instrument workspace within which the positioner 334 should be in a straightened condition for insertion or retraction of the instrument 110a into or out of the instrument workspace 324. The positioner operational envelope 1000 further includes a free movement region 1004 associated with the second constraint. The free movement region 1004 represents a region of the instrument workspace 324 within which the positioner 334 is able to move after the distal segment 346 of the positioner 334 is no longer disposed within the insertion/retraction region 1002 of the positioner operational envelope 1000.

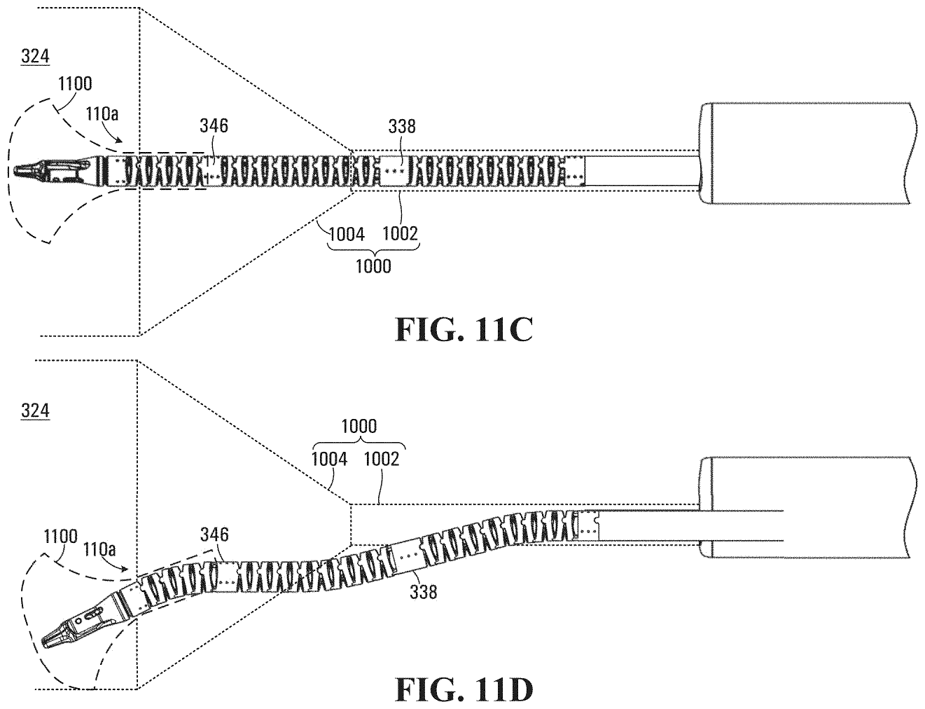

The operation of the positioner operational envelope 1000 is further described with reference to FIGS. 11A-11D, in which the instrument 110a is shown in various conditions. Referring to FIG. 11A, in this condition only the manipulator 332 and distal segment 346 are clear of the bore 302 of the insertion tube 108. While the distal segment 346 is already outside of the bore 302, a remainder of the positioner 334 is still constrained within the bore and cannot be moved. The distal segment 346 thus remains within the positioner insertion/retraction region 1002 of the positioner operational envelope 1000, which indicates that the positioner is currently constrained.

A manipulator operational envelope 1100 may also generated to define boundaries to movement of the distal end 352 of the manipulator 332 within the instrument workspace 324. In FIG. 11A, the manipulator 332 would be capable of movement within a manipulator operational envelope 1100 with respect to the distal segment 346.

Referring to FIG. 11B, the instrument 110a is shown in a condition where the manipulator 332, the distal segment 346, the intermediate segment 338, and a portion of the positioner 334 are all clear the bore 302 of the insertion tube 108. However the distal segment 346 remains just within the insertion/retraction region 1002 of the positioner operational envelope 1000, indicating that the positioner 334 remains constrained by the bore 302 of the insertion tube 108. The manipulator is still free to move within the manipulator operational envelope 1100, which in FIG. 11B has been advanced further into the instrument workspace 324.

Referring to FIG. 11C, the instrument 110a is shown in a condition where the distal segment 346 is well clear of the insertion/retraction region 1002 of the positioner operational envelope 1000. In this condition, the distal segment 346 is only constrained by the free movement region 1004 of the positioner operational envelope 1000. The positioner 334 is able to move within the respective free movement region 1004 of the positioner operational envelope 1000 and the manipulator 332 is able to move within the manipulator operational envelope 1100.

Referring to FIG. 11D, the instrument 110a as shown in FIG. 11C is now shown in a bent condition. Since the distal segment 346 is well clear of the insertion/retraction region 1002 of the positioner operational envelope 1000, the intermediate segment 338 and adjacent segments 340 and 342 may now move outside of the insertion/retraction region 1002 of the positioner operational envelope 1000. The distal segment 346 of the positioner 334 remains within the free movement region 1004 of the positioner operational envelope 1000. The intermediate segment 338 and adjacent segments 340 and 342 need not remain within the free movement region 1004 of the positioner operational envelope 1000. The positioner operational envelope 1000 thus defines boundaries to movement of the distal segment 346 of the positioner 334 within a portion of the instrument workspace 324.

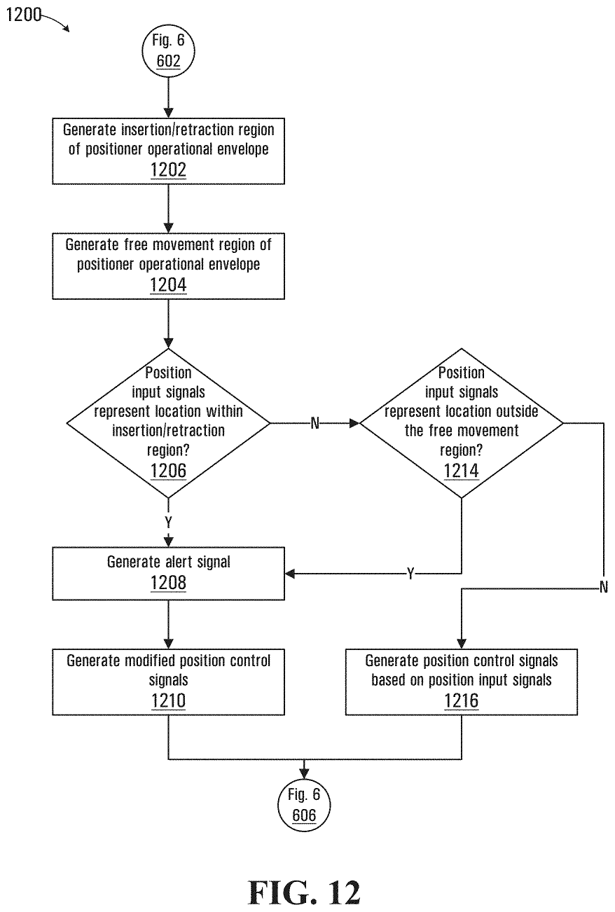

Referring to FIG. 12, a process embodiment that includes blocks of codes for causing the workstation processor circuit 114 to implement the positioner operational envelope 1000 while generating the position control signal is shown at generally at 1200. The process 1200 replaces block 604 in the process 600. The process 1200 begins at block 1202, which directs the microprocessor 400 of the workstation processor circuit 114 to generate the insertion/retraction region 1002 of the positioner operational envelope 1000. In this embodiment the insertion/retraction region 1002 is a cylindrical region within the instrument workspace 324 and may be generated as a three-dimensional cylindrical region in the fixed reference frame 700 (X₀, Y₀, Z₀) for the system 100a shown in FIG. 7A. In other embodiments the instrument 110 may be one of a set of articulating instruments of differing instrument types used in a surgical procedure and the positioner operational envelope 1000 may be a pre-defined positioner operational envelope for all differing instrument types. Alternatively the positioner operational envelope 1000 may be a pre-defined positioner operational envelope selected based on the instrument type. In other embodiments the positioner operational envelope 1000 may be established based at least in part on surgical data associated with a target operational site or based at least in part on surgical data provided by a scan of the target operational site. In some embodiments the positioner operational envelope 1000 may be based at least in part on operator input.

Block 1204 then directs the microprocessor 400 to generate the free movement region 1004 of the positioner operational envelope 1000. In this embodiment the free movement region 1004 is a frustoconical region extending outwardly from the distal end of the insertion/retraction region 1002. The free movement region 1004 may also be generated as a three-dimensional conical region in the fixed reference frame 700 (X₀, Y₀, Z₀).

In one embodiment the positioner operational envelope 1000 may be generated in the instrument workspace 324 and mapped from the instrument workspace into the input workspace 522. The generating of the alert signal at block 1208 may thus involve generating the alert signal when the position input signals received at the input device 112 represent a position in the input workspace that lies on or is outside the positioner operational envelope mapped to the input workspace.

The process 1200 then continues at block 1206, which directs the microprocessor 400 to determine whether the position input signals (received at block 602 of the process 600 in FIG. 6) represent a location in the instrument workspace 324 that is within the cylindrical region associated with the insertion/retraction region 1002. If the input signals represent a location that is within the cylindrical region, the microprocessor 400 is directed to block 1208. Block 1208 directs the microprocessor 400 to generate an alert signal to alert the operator that the position input signals represent a position in the instrument workspace 324 that lie on or outside the positioner operational envelope 1000.

In one embodiment, the input device 112 may deliver haptic feedback to the operator's hand in response to the operator alert signal. The alert signals may thus be in the form of a haptic feedback signal transmitted back to the input device 112 via the USB interface 404. The input device 112, on receiving the haptic feedback signal causes a haptic force or vibration to be generated via the hand controller 122 that alerts the operator to the alert condition. Alternatively or additionally, the microprocessor 400 may generate display signals that cause an alert to be displayed on the display 120 as an overlay image shown at 130 in FIG. 1.

Block 1210 then directs the microprocessor to generate modified position control signals based on the input signals received and the insertion/retraction region 1002 of the positioner operational envelope 1000. For example, if the distal segment 346 is still within the insertion/retraction region 1002 of the positioner operational envelope 1000, the prior position control signals may be maintained so as to prevent actuation of the positioner 334 that would cause bending.

As another example, if the instrument 110a is currently being withdrawn from the instrument workspace 324, the position control signals may be generated to cause a bent positioner 334 to be straightened. Referring back to FIG. 11D, block 1210 may direct the microprocessor 400 to determine that the position input signals received from the input device 112 are associated with a retraction of the distal segment 346 from the instrument workspace 324. In this case, block 1210 directs the microprocessor 400 to determine whether the received position input signal represents a physical position of the distal segment 346 associated with the positioner 334 being in a bent condition. If the positioner 334 is in a bent condition, block 1210 directs the microprocessor 400 to generate modified position control signals to cause the positioner 334 to be straightened while being retracted into the insertion/retraction region 1002.

Similarly, block 1210 may direct the microprocessor 400 to determine whether the received position input signal represents a physical position of the distal segment 346 that lies on or outside the positioner operational envelope 1000. If this is the case, block 1210 directs the microprocessor 400 to generate modified position control signals that constrain movements of the distal segment 346 to be within the positioner operational envelope 1004.

Block 1210 replaces block 604 in the process 600 and the modified control signals are transmitted to the instrument processor circuit 118 via the motion control interface 408. The instrument processor circuit 118 thus receives a modified position control signal and reacts accordingly when actuating the positioner 334.

If at block 1206, the position input signals represent a location that is not within the cylindrical region associated with the insertion/retraction region 1002, the microprocessor 400 is directed to block 1214. Block 1214 directs the microprocessor 400 to determine whether the position input signals represent a location in the instrument workspace 324 that is outside the conical region associated with the free movement region 1004. If the location is outside the free movement region 1004, block 1214 directs the microprocessor 400 to block 1208, which generates an alert signal generally as described above. Block 1210 then generates modified position control signals that maintain the physical position of the distal segment 346 inside the free movement region 1004 of the positioner operational envelope 1000. As described above, the alert signal may initiate haptic feedback, a displayed alert, or an audible alert. Block 1210 then directs the microprocessor 400 to transmit the modified control signals to the instrument processor circuit 118 via the motion control interface 408. The workstation processor circuit 114 responds by maintaining the current position of the distal segment 346 of the positioner 334.

If at block 1214 the location based on the position input signals remains within the free movement region 1004, block 1214 directs the microprocessor 400 to block 1216. Block 1216 directs the microprocessor 400 to generate position control signals based on the received position input signals and directs the microprocessor to transmit the position control signals to the instrument processor circuit 118 via the motion control interface 408. The workstation processor circuit 114 responds by actuating the distal segment 346 of the positioner 334 to move to the position represented by the position input signals.

The process 1200 thus causes position input signals that are not within the positioner operational envelope 1000 to be handled differently such that the operator is alerted to the undesirable movement and also prevents the movement from being effected. In instances where operator input received at the hand controller 122 of the input device 112 may result in damage to the instrument (such as when there is an attempt to retract a bent instrument), the instrument processor circuit 118 intercedes to force the straightening of the positioner 334 prior to being retracted in to the bore 302 of the insertion tube 108.

In some embodiments the instrument processor circuit 118 may be configured to generate display signals for displaying a view of the instrument workspace on the display 120 as shown in FIG. 1 at 120. The generated display signals may include display signals that cause an overlay image representing the positioner operational envelope 1000 to be displayed. The positioner operational envelope 1000 may be displayed as a colored or shaded region or an outline of the region, for example.

The process 1200 described above thus causes generation of an alert when the distal segment 346 of the positioner 334 would be positioned outside the positioner operational envelope 1000 based on current input signals being received at the input device 112. The manipulator operational envelope 1100 may be treated in the same manner and an alert signal generated by the microprocessor 400 when the orientation input signals represent an orientation in the instrument workspace 324 that would cause the distal end 352 of the manipulator 332 or the end effector 316 to lie on or outside the manipulator operational envelope 1100.

The instrument 110a may be one of a set of articulating instruments of differing instrument types used in a surgical procedure and the manipulator operational envelope 1100 may be a pre-defined manipulator operational envelope for all differing instrument types. Alternatively the manipulator operational envelope 1100 may be a pre-defined manipulator operational envelope selected based on the instrument type. In other embodiments the manipulator operational envelope 1100 may be established based at least in part on surgical data associated with a target operational site or based at least in part on surgical data provided by a scan of the target operational site. In some embodiments the manipulator operational envelope 1100 may be based at least in part on operator input.

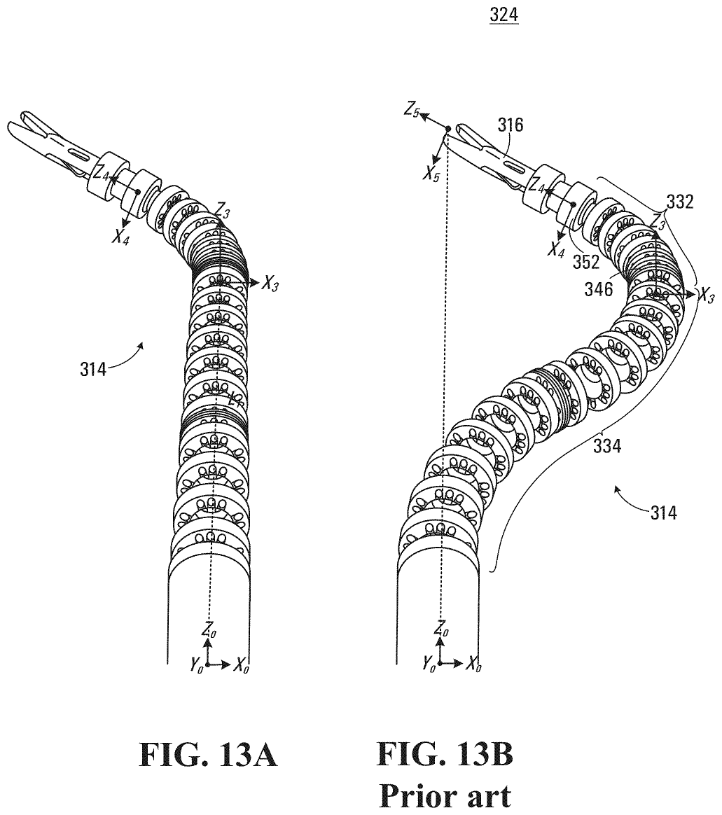

FIGS. 13A and 13B show different poses of the positioner 334 and manipulator 332 of the instrument 110a that highlight the differences between the control model described in the background section and the control mode disclosed in the above embodiments. In both of these Figures, the position of the hand controller 122 is assumed to be aligned with the z₁-axis in input workspace 522 (FIG. 5) and the orientation of the hand controller is rotated to the left about the y₁-axis.

Referring to FIG. 13A, the distal segment 346 of the positioner 334 in instrument workspace 324 is located at a position Z₃-X₃, which due to the hand controller 122 being aligned with the z₁-axis in input workspace 522 is located on the Z₀ axis in instrument workspace 324. The position of the distal segment 346 thus corresponds to the position of the hand controller 122 in input workspace 522. The manipulator 332 is bent to the left based on the orientation of the hand controller 122 to place the distal end 352 and end effector 316 at orientations X₄-Z₄ and X₅-Z₅ respectively. FIG. 13A is thus representative of the instrument 110a being controlled in accordance with the disclosed embodiments herein.

Referring to FIG. 13B, the end effector 316 is located at a position Z₅-X₅, which corresponds to the position of the hand controller 122 being aligned with the z₁-axis in input workspace 522. The end effector 316 is also oriented based on the orientation of the hand controller 122 to place the distal end 352 and end effector 316 at orientations X₄-Z₄ and X₅-Z₅ respectively. Thus, while the orientation of the end effector 316 is the same in both FIG. 13A and FIG. 13B, the actual position of the end effector X₅-Z₅ differs. In FIG. 13A, the end effector X₅-Z₅ is bent off the Z₀-axis in response to the orientation of the hand controller 122.

Additionally in FIG. 13B, the distal segment 346 of the positioner 334 in instrument workspace 324 is located at a position Z₃-X₃, which is calculated by the instrument processor circuit 118 to place the end effector 316 at the position in instrument workspace 324 corresponding to the position of the hand controller 122 in input workspace 522. The distal segment 346 is thus located to the right of the Z₀-axis. FIG. 13A is thus representative of the instrument 110a being controlled in accordance with the control mode described in the background section.

In one embodiment, the instrument processor circuit 118 may be responsive to a positioning mode change signal to change the control mode between the FIG. 13A and FIG. 13B control modes. For example, when the positioning mode change signal is received while in the mode depicted in FIG. 13A, the instrument processor circuit 118 may be configured to discontinue control based on the separate position control signals and the manipulation control signals. The processor circuit 118 would thus combine the position input signals and the orientation input signals received from the input device 112 to generate a desired position and orientation of the distal end distal end 352 of the manipulator or the end effector 316. Additionally, the instrument processor circuit 118 may also be configured to perform an inverse kinematic computation on the desired position and orientation of the distal end distal end 352 of the manipulator to determine a position for the distal segment distal segment 346 of the positioner 334. The instrument processor circuit 118 would further determine actuation parameters for the positioner 334 and the manipulator 332 associated with the desired position and orientation of the distal end 352 of the manipulator.

Each of the control modes of FIG. 13A and FIG. 13B have their respective advantages. The control mode in accordance with the embodiments disclosed herein has an advantage of controlling the location of the distal segment 346 of the positioner 334. The separate orientation control of the manipulator 332 may be better suited for performing some operations. The control mode of FIG. 13B may be advantageous when the system 100 is operating autonomously by the processor circuit 118 without input via the input device 112.

While specific embodiments have been described and illustrated, such embodiments should be considered illustrative only and not as limiting the disclosed embodiments as construed in accordance with the accompanying claims.

What is claimed is:

1. A method for controlling an articulating surgical instrument, the instrument comprising a manipulator and a positioner, the positioner being actuable to position a distal segment of the positioner within an instrument workspace, the manipulator attached to the distal segment of the positioner and including a distal end configured for mounting an operational tool for performing an operation within the instrument workspace, the manipulator being actuable to manipulate the distal end of the manipulator within the instrument workspace, the method comprising:
receiving input signals at a processor circuit, the input signals including:
position input signals representing a position within an input workspace; and
orientation input signals representing an orientation within the input workspace;
causing the processor circuit to generate position control signals for actuating the positioner to move the distal segment within the instrument workspace to a physical position represented by the position input signals; and
causing the processor circuit to generate manipulation control signals based on the orientation input signals for actuating the manipulator to orient the distal end within the instrument workspace.

2. The method of claim 1 wherein receiving input signals comprises receiving input signals from an autonomous controller processor circuit operably configured to autonomously generate the position input signals and the orientation input signals.

3. The method of claim 1 wherein receiving input signals comprises generating the input signals in response to movements of an operator's hand.

4. The method of claim 3 wherein generating the input signals in response to movements of an operator's hand comprises at least one of:
receiving movement signals from a sensor disposed to monitor free movements of an operator's hand within an input region;
receiving movement signals from a movement sensor grasped or attached to the operator's hand, the movement sensor being responsive to free movements of the operator's hand; or
receiving movement signals from a virtual reality headset worn by an operator.

5. The method of claim 4 further comprising causing the processor circuit to process the movement signals to generate the position input signals and orientation input signals.

6. The method of claim 5 wherein causing the processor circuit to process the movement signals comprises filtering the free movements of the operator's hand to extract movements of an operator's digits with respect to one of a palm of the operator's hand or a wrist of the operator's hand.

7. The method of claim 3 wherein generating the input signals comprises:
generating the position input signals in response to translations of the operator's hand in three translational degrees of freedom within the input workspace; and
generating the orientation input signals in response to rotations of the operator's hand in at least two rotational degrees of freedom within the input workspace.

8. The method of claim 7 wherein generating the position input signals comprises:
kinematically transmitting translational movements of the operator's hand in three translational degrees of freedom via a first kinematic structure to a plurality of encoders operable to produce translational movement signals; and
processing the translational movement signals to generate the position input signals.

9. The method of claim 7 wherein generating the orientation input signals comprises:
kinematically transmitting orientation movements of the operator's hand in the at least two rotational degrees of freedom via a second kinematic structure to a plurality of encoders operable to produce orientation signals; and
processing the orientation signals to generate the orientation input signals.

10. The method of claim 1 wherein the positioner comprises a first plurality of segments extending between a bulkhead segment and the distal segment, the first plurality of segments being selectively actuable by transmitting actuation forces via a first plurality of control wires extending through the first plurality of segments, and wherein actuating the positioner comprises generating the position control signals to selectively actuate the first plurality of control wires to cause respective movements of the first plurality of segments to position the distal segment at the physical position.

11. The method of claim 10 wherein the manipulator comprises a second plurality of segments extending between the distal segment of the positioner and the distal end of the manipulator, the second plurality of segments being moveable in response to transmitting actuation forces delivered via a second plurality of control wires extending through the first plurality of segments and through the second plurality of segments, and wherein actuating the manipulator comprises generating manipulator control signals to selectively actuate the second plurality of control wires to cause respective movements of the second plurality of segments to orient the distal end within the instrument workspace.

12. The method of claim 10 wherein the first plurality of segments comprise a plurality of adjacently stacked vertebra extending between the bulkhead segment and the distal segment and wherein the control wires are coupled to the distal segment and wherein selectively actuating the first plurality of control wires comprises actuating the first plurality of control wires to cause the distal segment to move to position the distal segment at the physical position, and wherein each vertebra is coupled to move in at least one of a pitch axis and a yaw axis with respect to adjacent vertebra and wherein the actuation forces delivered via the first plurality of control wires cause the respective vertebra to be angled with respect to each other to bend the instrument in a continuous arc.

13. The method of claim 12 wherein the first plurality of segments comprise a plurality of elongate segments coupled together by respective joints.

14. The method of claim 1 wherein the positioner is coupled to a rigid shaft and wherein:
the position input signals comprise an insertion depth position within the input workspace; and
actuating the positioner to move the distal segment within the instrument workspace comprises causing the rigid shaft to be advanced or retracted in response to the insertion depth position.

15. The method of claim 14 wherein the rigid shaft is received in a bore of an insertion tube and wherein the instrument workspace extends outwardly from an end of the insertion tube.

16. The method of claim 15 wherein causing the rigid shaft to be retracted comprises causing the rigid shaft to be retracted such that the distal segment of the positioner is disposed proximate the end of the insertion tube and the manipulator remains extending outwardly from the end of the insertion tube and remains capable of movement with respect to the distal segment.

17. The method of claim 1 further comprising:
generating a positioner operational envelope defining boundaries to movement of the distal segment of the positioner within a portion of the instrument workspace; and
generating an alert signal when the position input signals represent a position in the instrument workspace that lie on or outside the positioner operational envelope.

18. The method of claim 17 wherein receiving input signals comprises causing an input device to generate the input signals in response to movements of an operator's hand and further comprising delivering haptic feedback via the input device to the operator's hand in response to the alert signal.

19. The method of claim 17 wherein generating the positioner operational envelope comprises generating the positioner operational envelope in the instrument workspace and mapping the positioner operational envelope from the instrument workspace to the input workspace and wherein generating the alert signal comprises generating the alert signal when the position input signals represent a position in the input workspace that lies on or is outside the positioner operational envelope mapped to the input workspace.

20. The method of claim 17 wherein the processor circuit is operably configured to generate display signals for displaying a view of the instrument workspace and wherein generating the display signals comprises generating display signals including an overlay image corresponding to the positioner operational envelope.

21. The method of claim 17 wherein the positioner operational envelope comprises an insertion/retraction region that represents a region of the instrument workspace within which the positioner should be constrained in a straightened condition for insertion or retraction of the instrument into or out of the instrument workspace.

22. The method of claim 21 wherein the positioner operational envelope further comprises a free movement region that represents a region of the instrument workspace within which the positioner is able to move after the positioner is disposed outside of the insertion/retraction region of the positioner operational envelope.

23. The method of claim 17 wherein the articulating surgical instrument comprises one of a set of articulating instruments of differing instrument types used in a surgical procedure and wherein the positioner operational envelope comprises at least one of one of:
a pre-defined positioner operational envelope for all differing instrument types;
a pre-defined positioner operational envelope selected based on the instrument type;
a positioner operational envelope based at least in part on surgical data associated with a target operational site;
a positioner operational envelope based at least in part on surgical data provided by a scan of the target operational site; or
a positioner operational envelope based at least in part on operator input.

24. The method of claim 17 further comprising:
generating a manipulator operational envelope defining boundaries to movement of the distal end of the manipulator within a portion of the instrument workspace; and
generating an alert signal when the orientation input signals represent an orientation in the instrument workspace that would cause the distal end of the manipulator or the operational tool to lie on or outside the manipulator operational envelope.

25. The method of claim 24 wherein the articulating surgical instrument comprises one of a set of articulating instruments of differing instrument types used in a surgical procedure and wherein the manipulator operational envelope comprises at least one of one of:
a pre-defined manipulator operational envelope for all differing instrument types;
a pre-defined manipulator operational envelope selected based on the instrument type;
a manipulator operational envelope based at least in part on surgical data associated with a target operational site;
a manipulator operational envelope based at least in part on surgical data provided by a scan of the target operational site; or
a manipulator operational envelope based at least in part on operator input.

26. The method of claim 21 wherein in response to receiving position input signals that are associated with a retraction of the distal segment from the instrument workspace, further comprising:
causing the processor circuit to determine whether the position input signals represent a physical position of the distal segment associated with the positioner being in a bent condition; and
causing the processor circuit to generate modified position control signals to cause the positioner to be straightened while being retracted into the insertion/retraction region of the instrument workspace.

27. The method of claim 17 further comprising causing the processor circuit to determine whether the position input signals represent a physical position of the distal segment that lies on or outside the positioner operational envelope and wherein causing the processor circuit to generate the position control signals comprises causing the processor circuit to generate modified position control signals that constrain movements of the distal segment to be within the positioner operational envelope.

28. The method of claim 1 further comprising in response to receiving a positioning mode change signal, causing the processor circuit to:
combine the position input signals and the orientation input signals to generate a desired position and orientation of the distal end of the manipulator; and
perform an inverse kinematic computation on the desired position and orientation of the distal end of the manipulator to determine a position for the distal segment of positioner and actuation parameters for the positioner and the manipulator associated with the desired position and orientation of the distal end of the manipulator.
